(19) United States
(12) Patent Application Publication
(10) Pub. No.: US 2009/0148835 A1
(43) Pub. Date: Jun. 11, 2009

(54) METHOD FOR IDENTIFYING THE ORIGIN OF A COMPOUND BIOLOGICAL PRODUCT

(75) Inventors: Vanessa M. Cave, Dunedin (NZ); Allan Muirhead Crawford, Dunedin (NZ); Kenneth Grant Dodds, Dunedin (NZ); Helen Catherine Mathias, Dunedin (NZ); Grant Henry Shackell, Dunedin (NZ)

Correspondence Address: KNOBBE MARTENS OLSON & BEAR LLP, 2040 MAIN STREET, FOURTEENTH FLOOR, IRVINE, CA, 92614, US

(73) Assignee: AgResearch Limited, Hamilton (NZ)

(21) Appl. No.: 11/917,904

(22) PCT Filed: Jun. 19, 2006
(86) PCT No.: PCT/NZ2006/000158

§ 371 Date: Jul. 23, 2008

(30) Foreign Application Priority Data
Jun. 17, 2005 (NZ) 537318

(51) Int. Cl. C12Q 1/68

(52) U.S. Cl. 435/6

(57) ABSTRACT

The present invention relates to an identification method. In particular, a method for identifying the origin of a compound biological product, including the batch of origin, but also in some cases the actual biological sources of a compound biological product.

### TECHNICAL FIELD

The present invention relates to an identification method. In particular, a method for identifying the origin of a compound biological product, including the batch of origin, but also in some cases the actual biological sources of a compound biological product.

### BACKGROUND ART

Biological products have an in-built, unique identifier (DNA) that cannot be altered, and which can potentially be used to verify traceability systems. The value of DNA as a unique identifier of individual production animals lies in the fact that only clones (including identical twins) and some inbred crosses can have DNA profiles that are the same.

DNA analysis is widely used in a number of traceability and identification applications which require unequivocal identification of a particular species, strain or individual.

As a result of recent human health scares relating to disease, there is increasing demand for tracing meat and meat products. One example that has increased the demand for meat traceability is Bovine Spongiform Encephalopathy (BSE), a disease in cattle that has been linked to variant Creutzfeld Jacobs Disease (vCJD) in humans. Currently traceability relies on the integrity of an inventory trail, which although auditable is difficult to verify unequivocally.

Further, there is considerable consumer concern about the introduction of genetically modified foods and a desire to know where foods originate. DNA tests can be used to assure the species, origin and GMO status of food products.

However, when primary food items are reduced into saleable portions, the exact origin of those portions is often lost which means specific product information and potential value is also lost.

An increasing number of countries are presently implementing or developing full traceability requirements for meat products, for example the Japanese beef traceability law and the European animal tagging system.

A number of traceability systems are also currently being used which utilise DNA, such as that described in WO 00/61802. Such methods are typically used to trace saleable meat cuts through the product chain to the farm and animal of origin.

However, such traceability techniques typically involve a single meat cut or single DNA sequence being traced, the test being whether the DNA of the meat unambiguously matches the DNA of either one specific carcass or a species/strain specific DNA sequence. Given the expense, complexities involved and time constraints, such methods cannot be readily applied to compound meat products such as sausage meat or mince patties. In most processed compound meat products, the actual number of individuals contributing to the mixture, their relative concentrations within the mixture and their genotype is unlikely to be known. Therefore, the DNA profile of a compound meat product is much more difficult to interpret than the DNA profile of an individual.

The only current systems for tracing compound meat products are paper based, and can only supply batch information about the time, date and place of manufacture of the product.

Intentionally mixing DNA, usually in equal proportions, has been used as a laboratory tool to reduce genotyping effort in population studies to determine, for example, the association of allele frequencies with traits (e.g. Daniels et al., American Journal of Human Genetics, 62, 1189-1197. 1998) and biodiversity (Hillel et al., Genetics Selection Evolution, 35, 533-557. 2003).

Forensic science also deals with mixed DNA samples, although the mixtures typically have a relatively small number of individuals (typically less than 5) and the analysis merely aims to include (or eliminate) the presence of specific individuals within the mixture rather than actually identify an individual.

Egeland, Dalen and Mostad, International Journal of Legal Medicine, 117, 271-275, (2003) suggested that under certain assumptions, when profiles contain relatively small numbers of individuals, knowledge of allele frequencies in the population being investigated can be used to estimate of the number of individuals contributing to a mixture, even with bi-allelic loci. However, large numbers of loci (100-1000) are required to obtain accurate estimates.

However, despite the advances outlined in the work of Daniels et al; Hillel et al; and Egeland et al, in further studies by Dodds and Shackell, XXII^{nd} International Biometric Conference, Cairns, Australia, pp. 433, (2004) it has been found that it is difficult to identify the number of diploid individuals in a mixture with microsatellite panels typically used for parentage tests when there were more than five or six contributing individuals. This methodology used fewer assumptions than the method of Egeland et al described above.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein; this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a method for identifying the batch origin of a compound biological product, including the steps of either:
(i) obtaining from a reference-sample at least one genetic profile of at least one individual contributor to a batch of compound biological product; or
(ii) obtaining from a reference-sample a genetic profile of a compound biological product from a batch wherein the genetic profile of individual contributors may or may not be known;
   the method further characterised by the steps of:
   a) recording the genetic profiles from i) or ii) to create reference-sample records and linking these to batch information for the purposes of later identification;
   b) taking a test-sample of the compound biological product to be identified,
   c) optionally, obtaining a component-sample by reducing the test-sample into one or more component particles (i.e. individual contributors);
   d) obtaining at least one genetic profile of either the test-sample at step b) or component-samples at step c); and
   e) comparing the profile(s) from step d) to the genetic profiles from steps (i) or (ii) of the reference-sample records, for at least one match, so as to identify the batch origin of the compound biological product of the test-sample.

It should be appreciated by those skilled in the art that the present invention can be performed manually or via a suitably programmed computer.

In general the genetic profiles may be recorded in a database.

In preferred embodiments the genetic profiles may be may be recorded in a computer database.

According to a further aspect of the present invention there is provided a method substantially as described above wherein the method includes the further step of applying a mathematical formula using information on genetic inheritance to assign probabilities for the misidentification by DNA analysis of individuals in a sample, due to relatedness of one or more of those individuals in the reference-sample.

The mathematical formula for assessing a probability that the profiles of two unrelated individuals matching is

$${MP}_{⋃} = {\prod }_{j = 1}^{k} [ 2 {( {\sum }_{i = 1}^{{v}_{j}} {p}_{ij}^{2} )}^{2} - {\sum }_{i = 1}^{{v}_{j}} {p}_{ij}^{4} ]$$

Wherein pᵢⱼ refers to the ith allele of the jth marker;
Vⱼ is the number of alleles of the jth marker, and
k is the number of markers.

Similar formulas are available for specified amounts of relatedness and are well known to those proficient in the art.

According to a still further aspect of the present invention there is provided a method substantially as described above wherein there is provided a method for identifying the batch origin of a compound biological product, including the steps of either:
(i) obtaining from a reference-sample at least one genetic profile of at least one individual contributor to a batch of compound biological product; or
(ii) obtaining from a reference-sample a genetic profile of a compound biological product from a batch wherein the genetic profile of individual contributors may or may not be known;
iii) inputting the genetic profile information into a computer database, after obtaining a genetic profile from steps (i) or (ii);
   the method further characterised by the steps of:
   a) recording the genetic profiles from i) or ii) to create reference-sample records and linking these to batch information for the purposes of later identification;
   b) taking a test-sample of the compound biological product to be identified,
   c) optionally, obtaining a component-sample by reducing the test-sample into one or more component particles (i.e. individual contributors);
   d) obtaining at least one genetic profile of either the test-sample at step b) or component-samples at step c);
   e) comparing the profile(s) from step d) to the genetic profiles from steps (i) or (ii) of the reference-sample records, for at least one match, so as to identify the batch origin of the compound biological product of the test-sample; and
   f) inputting the genetic profile information into a computer database, after obtaining a genetic profile at step d) and wherein step e), insofar as it relates to a genetic profile of a test sample (as opposed to a component sample) is undertaken by a suitably programmed computer which can access said database.

According to another aspect of the present invention there is provided a method substantially as described above wherein the computer is programmed to assess the posterior probabilities of the sample being derived from each of the reference-sample sources.

According to a further aspect of the present invention there is provided a method substantially as described above wherein the computer is programmed to assess the posterior probabilities of the sample being derived from each of the reference-sample sources via a statistical classification assessment. Suitable statistical classification assessments will be well known to those skilled in the art.

According to a still further aspect of the present invention there is provided a method substantially as described above wherein the computer is programmed to assess the posterior probabilities of the sample being derived from each of the reference-sample sources via a supervised machine learning assessment.

Suitable supervised machine teaching techniques will be well known to those skilled in the art.

According to another aspect of the present invention there is provided a method substantially as described above wherein the number of genetic profiles required to be obtained from individual contributors, where it is envisaged only one component particle will subsequently be available for testing, is determined after assigning a level of probability, of incorrectly failing to identify a match.

According to a further aspect of the present invention there is provided a method substantially as described above wherein the assigned level of probability is set at 10⁻⁵.

According to a still further aspect of the present invention there is provided a method substantially as described above wherein the level of probability is determined by applying the formula:

$$\begin{matrix}P ( {{⋃}_{i = 1}}^{{k}_{r}} {E}_{i} ) = {\sum }_{i = 1}^{{k}_{r}} {E}_{i} - {\sum }_{i < j} \sum {E}_{i} {E}_{j} + {\sum }_{i < j < k} \sum \sum {E}_{i} {E}_{j} {E}_{k} - … \\ = \frac{{k}_{r}}{n} - ( \begin{matrix}{k}_{r} \\ 2\end{matrix} ) \frac{1}{{n}^{2}} + ( \begin{matrix}{k}_{r} \\ 3\end{matrix} ) \frac{1}{{n}^{3}} - … \\ = {\sum }_{u = 1}^{{k}_{r}} - {( - 1 )}^{u} ( \begin{matrix}{k}_{r} \\ i\end{matrix} ) \frac{1}{{n}^{u}}\end{matrix}$$

wherein variable ‘P’ is the probability of the test-sample matching the reference-sample;
and wherein variable ‘n’ is the maximum number of individuals likely to be represented in the batch,
and wherein kr is the number of component particles from the reference samples;
and Ei denotes that there is a match between a test sample and the ith reference sample;
and i, j, k and u are indexing variables associated with the component particles.

According to another aspect of the present invention there is provided a method substantially as described above wherein the number of genetic profiles required from individual contributors, where it is envisaged more than one component particle will be subsequently available for testing, is determined after assigning a level of probability, of correctly identifying a match, after a set number of unique genetic profiles derived from the test sample, are found to correspond to genetic profiles in the reference sample records, for a given batch.

According to a further aspect of the present invention there is provided a method substantially as described above wherein the set number is determined according to the estimated number of contributors to a batch and the likelihood of any of those individuals contributing to more than one batch.

According to a still further aspect of the present invention there is provided a method substantially as described above wherein the probability is set at 0.95.

According to another aspect of the present invention there is provided a method substantially as described above wherein the number of genetic profiles required to be obtained from individual contributors, where it is envisaged that more than one component particle will subsequently be available for testing, is determined after assigning a level of probability, of failing to identify a match.

According to a further aspect of the present invention there is provided a method substantially as described above wherein the assigned level of probability is set at 10⁻⁵.

According to a still further aspect of the present invention there is provided a method substantially as described above wherein the level of probability is set by the simulation steps of:
1) Generating a list of n contributor identifiers;
2) Generating a list of kᵣ results from the reference product, by sampling (with replacement) from the contributor identifiers;
3) Generating a list of kₜ results from the reference product, by sampling (with replacement) from the contributor identifiers;
4) Counting the number of unique identifiers that appear in both lists generated in steps 2 and 3;
5) Repeating steps 2-4 sufficiently many times to give a stable distribution of the counts in step 4; and
6) Converting the accumulated results from step 4 into probabilities.

According to another aspect of the present invention there is provided a computer which is programmed to identify the batch origin of a compound biological product from a computer database of reference-sample records linked to batch information via the steps of:
a) inputting information of at least one genetic profile obtained from a test-sample or component-sample into the computer;
b) comparing the genetic profile(s) from step a) against the appropriate genetic profiles of a reference-sample computer database;
c) calculating likelihoods of a match and converting them to statistical probabilities.

According to a still further aspect of the present invention there is provided a method substantially as described above wherein a computer storage medium which includes a program to perform a method as substantially described above.

According to a further aspect there is provided a method of determining batch origin for subsequent use with the above method, comprising the steps of either:
i) obtaining a reference-sample of at least one individual contributor to a batch of compound biological product; or
ii) obtaining a reference-sample of a compound of biological product wherein the genetic profile of individual contributors may
or may not be known;
iii) the method further characterised by the steps of:
   a) storing the reference-samples from a batch; and
   b) linking the reference-samples to batch information;
   c) assigning a probability before undertaking the methodology as substantially described above.

The term “database” as used herein refers to a structured set of data (i.e. genetic profiles) which is stored in a readily retrievable and secure location.

The term “computer database” is as used herein refers to a database which is stored in a computer or like device.

The term “batch” as used herein should generally be taken to mean a defined quantity of compound biological product, identified as being produced from components obtained from exactly the same set of biological sources at a specific time, date and place of manufacture. Batch production and recordal of batch information is standard practice within the food industry.

The term “match” as used here refers to a genetic profile derived from a test sample being found to correspond to a genetic profile of a reference-sample record. In some cases X number of genetic profiles must be derived from the test sample and these profiles must correspond to X number of reference sample records in order for there to be a match.

The term “compound biological product” refers to any product which includes a component from more than one discrete biological source. In general, the biological sources may be from animals, or part(s) thereof. Preferably, the compound biological product will be a food product.

The term “individuals” refers to animals, or parts thereof which are used in producing a compound biological product.

In preferred embodiments of the present invention the compound biological product will be a compound meat product, such as sausage meat, meat patties or the like.

For ease of reference, the term “compound biological product” may hereinafter be referred to as a compound meat product, such as ground beef. However, this should not be seen as limiting, as the present invention is applicable to animal products other than beef, and to compound biological products other than meat. For example, the present invention may be equally applicable in determining the composition and origin of components in other biological products such as processed foods including animal products therein, animal feed, or so forth.

In some preferred embodiments the component particles may be single grains or fibres of meat. In some further preferred embodiments the component particles may be individual cells.

The term “genetic profile” should generally be taken to refer to genetic information detailing one or more markers of interest for distinguishing individuals, or a group of individuals. A genetic profile can indicate the distribution of the alleles, or a number of polymorphic genetic markers, e.g. SNPs or microsatellites, or, can be information that indicates subtle changes in the pattern of allelic variation in samples that contain DNA or RNA from many individuals.

Throughout this specification, the term “allele” shall refer to a genetic variant of a genetic locus that is polymorphic.

As used herein, the term “locus” (pl. loci) refers to a position on a chromosome, gene or other DNA sequence.

As used herein, the term “marker” shall refer to an identifiable difference in nucleotide sequence at a known location, on a strand of DNA of an animal which is capable being used to distinguish individuals. The term “marker” includes: allelles, microsatellites, SNPs, which are polymorphic.

Throughout the specification, the term “polymorphic” refers to something having two or more distinct forms.

As used herein, the terms “DNA” and “RNA” include cRNA, genomic DNA or cDNA molecules, and may be single or double-stranded.

For ease of reference only, the terms “DNA” and “RNA” will now generally be referred to simply as “DNA”.

Throughout the specification the term “microsatellite” refers to a type of marker which comprises a short sequence of nucleotides that is repeated. For example, the microsatellite ATAATAATAATA is a repeat of the ATA nucleotide sequence. Where individuals have microsatellites of different lengths (i.e. more or less repeats), these are useful markers to distinguish individuals.

In preferred embodiments, DNA is obtained and then processed to obtain a genetic profile. In some embodiments, RNA may be used to obtain a profile for subsequent analysis by the method of the present innovation. In some embodiments, single nucleotide polymorphisms (SNPs) may be used to distinguish between different batches of a compound biological product.

As used herein, the term “single nucleotide polymorphic” or “SNP” refers to a single nucleotide which differs from that usually found at a locus.

In preferred embodiments, a reference-sample may be collected from either one or more individuals contributing to a batch of compound meat product, or collected from the batch of compound meat product upon manufacture, then stored for the purposes of later identification. In some embodiments a reference-sample may be collected from every individual known to contribute to a batch of compound ground product.

Upon analysis the reference-sample of a batch is expected to reveal the aggregate genetic profile indicative of one or more individual animal contributors as is required to be representative for that batch of origin (i.e. the genetic profile of a batch must distinguish the batch from the genetic profiles of other batches).

The term “test sample” as used herein refers to a sample taken from a compound biological product to be identified.

The term “batch information” as used herein refers to any unique combination of symbols or other information which can be stored for subsequent retrieval that is capable of distinguishing one batch from another batch so as to act as an identifier. In preferred embodiments the batch information may be an alpha-numeric identifier,

A compound meat product may be reduced to its component particles (i.e. individual contributors) by dismantling and dissecting out single grains or fibres of meat. Upon extracting the DNA from these single pieces of meat the genetic profile is scored and the piece of meat is deemed to have come from a single animal if the are no more than two alleles present at every microsatellite marker. If there are consistently more than two alleles present at some markers the sample is deemed to be contaminated

As used herein the term “reference-sample” in relation to a compound biological product means a sample of a batch of compound biological product taken at the time of manufacture which includes DNA representative of the batch.

As used herein the term “reference-sample” in relation to an individual animal means a DNA sample taken at some time prior to the dismantling of the carcass of the individual. For example, a reference-sample for an individual animal may be taken on the farm of origin or at the time of transport or at the time of slaughter. At all times a reference-sample for an individual is a DNA sample taken at a time when the animal can unambiguously be identified as an individual.

Throughout the specification, the term “component-sample” refers to a sample containing the DNA of a single individual that has been isolated and removed from a sample of a compound biological product.

For the purposes of identifying the contributors to a compound meat product the product must be dismantled into components that can only have originated from one individual. In order to be sure that a component-sample is not contaminated, the genetic profile must have at least one and no more than two peaks for each of the markers used. A sample that has three peaks at any marker is contaminated and the origin is then in dispute.

In some embodiments it may be possible to determine by inference that a component-sample comes from an individual if the genotypes of all the contributing individuals are known and the number of contaminated markers is very low and the individual has alleles that are unique and these are identifiable as non-contaminated markers.

In preferred embodiments ground meat products are dismantled by dissection into discrete pieces. In some embodiments this is achieved by dissection under nil or low magnification. In some other embodiments this is achieved by dismantling the product under a dissecting microscope. In further embodiments this may be achieved by cellular sorting technology. Examples of suitable sorting technology will be well known to those skilled in the art.

In preferred embodiments the compound meat sample is immersed in an organic solvent (typically, but not limited to, ethanol or chloroform or acetone or a combination of organic solvents) to disburse the fat content prior to dismantling the meat sample. After removing the sample from the solvent the individual fibres of meat are then separated and passed through several washes of a physiological buffer (typically, but not limited to, phosphate buffered saline or normal saline or Tris (Hydroxymethyl Aminomethane)/edta (ethylenediaminetetraacetic acid) to remove the solvent.

In some embodiments one or more of the washes may contain a low concentration of Proteinase K or another enzyme to assist with cleaning the surface of the component-sample by partial digestion.

The number of markers required to identify individual contributors will be dependent on several factors, including but not limited to the informativeness of the markers themselves (i.e. the uniqueness of the markers); the species of animal making up the compound meat product; and the degree of relatedness between individuals.

The genetic profiles may be obtained using any suitable techniques, including those standard molecular biology techniques presently known in the art. Suitable known techniques involve DNA extraction and preparation, and microsatellite genotyping to determine the distribution of the genetic markers.

Groups of the markers may preferably be compared (i.e. analysed) simultaneously using standard techniques known in the art, such as multiplex or parallel analysis systems.

References: Shuber et. al., 1995. A simplified procedure for developing multiplex PCRs Genome Research 5: 488-493; Henegariu et. al., 1997. Multiplex PCR: Critical parameters and step-by-step protocol BioTechniques 23(3) 504-511.

In preferred embodiments, the genetic profiles may be obtained from at least one, but preferably two or more, highly polymorphic microsatellite markers which may be able to be multiplexed (i.e. analysed together).

Using current technology, approximately 15 microsatellite markers chosen to be highly polymorphic and able to be multiplexed in groups of 4-6 may most preferably be used. However, this should not be seen as a limitation. In some cases where mixtures are known to contain small numbers of individuals, or when the markers are highly informative, a smaller set of markers can be used. In other cases a larger set (30-40) of markers with a lower level of polymorphism may be used, this may include microsatellites or SNP markers. In the future, improved marker technology and/or more informative markers may become available and may be used.

It is understood that with knowledge of the art multiplexing systems may be designed that group the markers in different groups and group sizes. Different multiplexes may used in some embodiments even though the aggregate marker group remains unchanged.

In preferred embodiments, the microsatellite markers of the test and reference samples may be analysed in either an ABI PRISM 3100 or ABI PRISM 3730 Genetic Analyser (Applied BioSystems) and scored with Genotyper v3.7 or Genemapper v3.0 software respectively (Applied BioSystems), to produce a genetic profile. However, this should not be seen as limiting as other DNA analysers and software may be used, and improved technology may become available in the future.

Both programmes generate a DNA ‘signal’ profile and allow the assignment of values to each DNA fragment for fragment size (a form of speed of migration in the capillary and the number of base-pairs of DNA in the fragment) which, following analysis are represented as peaks with their height and area expressed in relative fluorescence units (r.f.u). Each sample should comprise peak scores at all of the markers.

In preferred embodiments, when DNA profiles of mixtures are being analysed all peaks are considered in the analysis. This is because it is impossible to distinguish between allele peaks and stutter (an artefact of DNA genotyping) in a mixed sample containing many individuals.

Reference: Shackell et. al., 2005. Evaluation of microsatellites as a potential tool for product tracing of ground beef mixtures. Meat Science 70: 337-345

The term “peak score” refers to the peak height and area under the peak measured by the genotyping software at each DNA fragment size. In normal genotyping of individuals either one or two peaks (alleles) are seen at each marker. When a compound mixture is analysed, there will usually be more than two peaks at each marker and the total number of peaks may be higher than the number of alleles identified at that marker as stutter peaks are included in the genotype profile.

According to a further aspect of the present invention there is provided computer software adapted to implement a method for identifying the origin of a compound biological product.

In some embodiments, it may be known that the compound meat product can only have come from one of a small number of batches. DNA profiles of individual animals contributing to the compound meat product being tested can be compared against the DNA profiles of component particles isolated from the representative sample of the batch(es) being tested, to determine whether both samples match (i.e. come from the same batch).

The term “computer” as used herein refers to a device which includes a central processing unit or the like and an associated memory device.

In other embodiments, DNA profiles may be obtained from each batch of compound meat product as it is manufactured, with the DNA profiles being stored in a database for the purpose of later identification. When a comparison is desired, the DNA profiles obtained from a sample of compound meat product can be compared against the database to identify the originating batch of the sample.

In preferred embodiments the database may contain batch information on each reference-sample for the purposes of cross referencing during later identification.

The term “component particle” should generally be taken to refer to individual grains, or fibres, of tissue, which come from a single animal.

To obtain DNA profiles for separate individuals, the sample has to be reduced down into component particles of tissue. In preferred embodiments the sample may be reduced into component particles by dissecting the compound meat product.

In some cases where an animal contributing to a compound biological product has already been profiled, obtaining a profile may simply require access to a database record of the genetic profile for that animal.

In further embodiments of the present invention, the component particles may be reduced down to a single cell.

The isolation and extraction of single cells allows the present invention to be used in products where the size of the component particles is much smaller than those found in ground meat.

To confirm whether the component particles come only from a single animal, it is necessary to confirm that each particle has no more than two alleles for any given marker (i.e. the markers must not be contaminated).

In preferred embodiments of the present invention, the genetic profiles may be obtained in relation to a set of known animal microsatellite markers. For example, genetic profiles for bovine microsatellite markers may be obtained, however this should not be seen as limiting. Such markers are commonly used for parentage testing. Although, to date, until the present invention, it has been difficult to use microsatellites as a method of tracing individual animals in compound mixtures as described by Egeland, Dalen and Mostad, 2003 and Dodds and Shackell, 2004.

The microsatellite markers preferably used contain two base pair repeats, giving length variants which are a minimum of two base pairs from their nearest neighbours. Frequently, small amounts of fragments two, four or occasionally six base pairs smaller than the actual allele are also amplified, a phenomenon referred to as stutter.

When an uncontaminated sample from an individual is profiled there are no more than two major peaks seen at each locus. Major peaks represent the real alleles and stutter is a minor consideration because differences in peak height eliminate indecision in allele identification. However, when a mixture of individuals is genotyped it is not always possible to differentiate between low peaks due to stutter and the allele(s) of an individual making only a minor contribution to the mixture.

Therefore, when microsatellite markers are run for samples that contain a mixture of individuals, consideration has to be given to the likely presence of low peaks due to stutter, and their interaction with the alleles of individuals making only a minor contribution to the mixture.

Thus preferred embodiments of the present invention may have a number of advantages over the prior art which include:
a. a non-paper based system for tracing the origin of compound meat products.
b. A verifiable method for configuring the origin of a compound meat product from the meat product itself (i.e. the method is not reliant on packaging or associated with the meat products).

### BRIEF DESCRIPTION OF DRAWINGS

Further aspects of the present invention will become apparent from the following description which is given by way of example only and with reference to the accompanying drawings in which:

FIG. 1 shows allele profiles at a single marker. The top row shows all peaks overlaid. Rows 1-5 show genotypes for one homozygous (row 2) and four heterozygous (rows 1, 3, 4 and 5) contributors. Row 6 shows the genotype of a mixture containing meat from each of the five contributors in which all of the alleles are present. The contributors in rows 3 and 5 each have a unique allele and can be definitively assigned to the mixture; the other contributors cannot be separated.

FIG. 2 shows allele profiles of component particles dissected from a compound meat product at a single marker. The top row shows all peaks overlaid. Row 1 identifies a homozygous individual. Rows 2, 3 and 4 identify heterozygous individuals. Row 5 is contaminated and does not identify an individual animal.

FIG. 3 shows allele profiles of batches of the same contributing animals mixed in different proportions. The differences between batches show that mixtures can be identified from each other even when the same animals contribute to them.

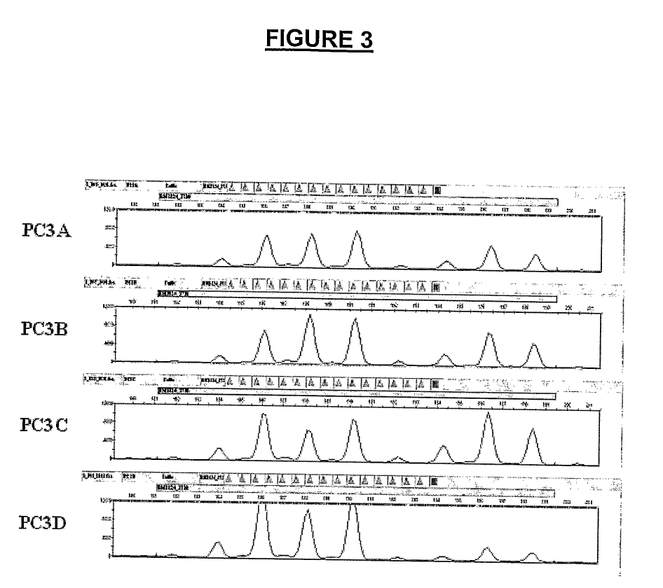

FIG. 4: shows a schematic example of how identifying some individual contributors in a meat patty can be used to decide whether or not that patty came from a batch of ground meat where every contributing animal is known. The batch shows that there were 10 contributing individuals. For patties 1 and 2 all three individuals can be identified in the batch and therefore those patties must have come from the batch (one individual contributor is identified in both patties). None of the individuals in patty 3 were in the batch and therefore the patty did not come from the batch. In patty 4, two individuals were in the manufacturing batch, but one was not. Since all contributors are known, patty 4 must have been contaminated with meat from another source.

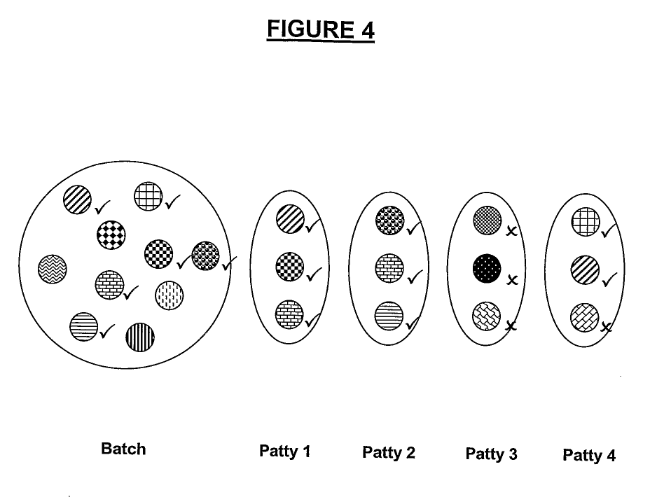

### BEST MODES FOR CARRYING OUT THE INVENTION

As defined above, in its primary aspect, the present invention is directed to a method for the identification of a compound food product and the subsequent identification of the batch of origin. The invention has particular application to compound meat product such as ground beef. However, this should not be seen as limiting the scope of the present invention to other compound biological products.

### Mathematical Modelling

A method was developed to determine the number of component particles (from single individuals) needed from the compound meat product being tested, (k) as well as from the representative sample of the batch being analysed (kₜ) to identify the batch of any given sample of compound meat product.

Assume that a batch is comprised of products from n individuals, in equal proportions (if there was a known unequal distribution of individuals that also could be modelled).

The batch being tested will be declared as the probable source of the test product if there is some minimum number, m, of matches between the test and reference-samples.

In preferred embodiments, this number (m) can be calculated to allow for an incorrect match between one animal and some other non-related animal, which is very rare, and to allow for individual animals that may contribute to more than one batch.

In preferred embodiments the line of supply of meat to be used for ground meat product will be known and the probability of a given number of animals all contributing to more than one batch can be modelled for the total number of animals likely to be represented in the batch. The data from this model can be used to calculate the number of samples that must be taken (m). These samples are then used to confirm or reject the batch as the source of trace patties (see FIG. 5).

The procedure is planned in such a way that if the test and reference-samples have the same source, at least this many matches will be observed with the desired probability (say 0.95).

In some embodiments, a sampling calculator (based on software developed with knowledge of the production and packaging of meat that is to be used for ground meat product) may be provided so that sampling decisions prior to manufacture and during trace procedures can predict the number of samples that must be taken at the point of manufacture and during dismantling of the product for identification purposes.

Reference: Vetharaniam, I., and Shackell, G. H., 2005. Software for evaluating sampling strategies and error rates in the identification of mixed-meat products. Proceedings of the New Zealand Society of Animal Production 65:102-106

The batch being tested will be declared as not the probable source if none of the trace samples matches any of the reference-samples, and the probability of this event occurring, given that the trace and reference have the same source, is sufficiently low. In preferred embodiments this value is set at 10⁻⁵.

In preferred embodiments, if the number of matches falls within the upper and lower bounds required to confirm or reject a match, further sampling would be used to try to increase the number of matches to a number above the upper threshold.

When there is only one trace sample, or one reference-sample the probability of that sample matching one of the samples from the other set can be calculated. For example, if kₜ=1, then the probability the trace sample matches one of the reference-samples is

$$\begin{matrix}P ( {{⋃}_{i = 1}}^{{k}_{r}} {E}_{i} ) = {\sum }_{i = 1}^{{k}_{r}} {E}_{i} - {\sum }_{i < j} \sum {E}_{i} {E}_{j} + {\sum }_{i < j < k} \sum \sum {E}_{i} {E}_{j} {E}_{k} - … \\ = \frac{{k}_{r}}{n} - ( \begin{matrix}{k}_{r} \\ 2\end{matrix} ) \frac{1}{{n}^{2}} + ( \begin{matrix}{k}_{r} \\ 3\end{matrix} ) \frac{1}{{n}^{3}} - … \\ = {\sum }_{u = 1}^{{k}_{r}} - {( - 1 )}^{u} ( \begin{matrix}{k}_{r} \\ i\end{matrix} ) \frac{1}{{n}^{u}}\end{matrix}$$

wherein variable ‘P’ is the probability of the test-sample matching the reference-sample;
and wherein variable ‘n’ is the maximum number of individuals likely to be represented in the batch,
and wherein kr is the number of component particles from the reference samples;
and Ei denotes that there is a match between a test sample and the ith reference sample;
and i, j, k and u are indexing variables associated with the component particles;
where Eᵢ is the event that the trace sample matches the ith reference-sample, and since the probability of matching u particular reference-samples is 1/n^{u}.

For situations with both kᵣ and kₜ greater than one, a simulation method can be used to provide the relevant probabilities. The simulation finds the probability of finding the specified number of matches (or no matches) for a range of values of kᵣ and kₜ. Usually, these two values would be set equal (giving higher efficacy at the same total number tested), but they may be different, for example when the number that can be tested from one of the samples is limited.

In one preferred embodiment or simulation method proceeds as follows:
1. Generate a list of n contributor identifiers;
2. Generate a list of k, results from the reference product, by sampling (with replacement) from the contributor identifiers;
3. Generate a list of kₜ results from the reference product, by sampling (with replacement) from the contributor identifiers;
4. Count the number of unique identifiers that appear in both lists generated in steps 2 and 3.
5. Repeat steps 2-5 sufficiently many times to give a stable distribution of the counts in step 4.
6. Convert the accumulated results from step 4 into probabilities.

For examples, in Trial 2 (discussed below) reference-samples were taken from seven of the contributors to a mixture. A test-sample was taken from the mixture and kₜ=15 and dismantled to obtain component-samples which were analysed. These 15 component-samples were identified as coming from eight different individuals, six from the reference-samples and two others. Therefore, n>9 (the seven known contributors and the two contributors in the test sample that were not one of these seven). We now suppose that the reference-samples were taken from the mixture and seven contributors identified. The most likely values for n and k, given rise to seven are (see Feller, 1968) n=9 and kᵣ=13 (i.e. we are supposing that there are nine contributors to the mixture, that we took 13 reference samples and found seven different individuals amongst these). Then, using the simulation method (described above) with 10 million replicates, we find the following probabilities:

| Number of matches | Probability |
|---|---|
| 0 | 0.0000008 |
| 1 | 0.0000688 |
| 2 | 0.0017678 |
| 3 | 0.0189980 |
| 4 | 0.0979797 |
| 5 | 0.2553562 |
| 6 | 0.3373779 |
| 7 | 0.2191299 |
| 8 | 0.0632687 |
| 9 | 0.0060522 |

The observed number of matches was six. The probability of at least two matches is 99.993%. The probability of no matches is less than 10⁻⁶.

In practice, n is unknown, so the calculations need to be repeated for a range of plausible values on a case by case basis to ensure that any statements made about exclusion are conservative.

Reference: Feller, 1968, An Introduction to Probability Theory and Its Applications, Volume 1, 3^{rd} ed., Wiley, New York, p 102

### Experimental Trials

### Protocol

A series of experiments were designed to investigate the potential for using microsatellite DNA genotyping technology to identify individuals from a compound meat product.

### DNA Preparation

All mixtures and samples were homogenised for 10-15 seconds at 11,000 rpm using a high speed disperser (Ultra Turrax, IKA). The homogeniser probe was dismantled and cleaned between every sample.

During homogenisation, a mass of connective tissue that accounted for up to 50% of the weight of the sample formed on the bottom of the homogeniser or in the mixing tube. This was removed and the weight subtracted from the initial sample weight.

An aliquot volume was then calculated from the net weight of each sample to give 25 mg of homogenised muscle and fat in a constant volume (15 ml) of TE buffer per assay.

DNA was extracted using a commercial extraction kit (DNeasy, Qiagen). Extracted samples were analysed in a NanoDrop ND-1000 Spectrophotometer (Nanodrop Technologies, Rockland, USA) to determine the DNA concentration and diluted to a concentration of 50 ng/μl. Samples containing <50 ng/μl were not diluted.

### Microsatellite Genotyping:

The method currently uses, but is not limited to 15 markers [AGLA293, BM1824, BM2113, ETH3, ETH10, ETH225, INRA23, MGTG4B, MGTG7, SPS115, TGLA53, TGLA122, TGLA126, TGLA263 and TGLA227]. However, it will be appreciated by those skilled in the art other markers may also be employed in the present invention.

Details of these markers are in the Public Domain and are available at the following websites:

http://lous.jouy.inra.fr/cqi-bin/lgbc/mapping/common/main.pl?BASE=cattle (for all except SPS115)
and http://www.projects.roslin.ac.uk/cdiv/markers.html (for SPS115).

DNA was amplified by Polymerase Chain Reaction using the following conditions: 94° C. for 30 seconds, 59° C. for one minute, 72° C. for 30 seconds, cycled 35 times with an MgCl₂ concentration of 3.0 mM, in an MJ Research thermal cycler.

The amplified markers were analysed in either an ABI PRISM 3100 or ABI PRISM 3730 Genetic Analyser (Applied BioSystems) and scored with Genotyper v3.7 or GeneMapper v3.0 genotyping software respectively (Applied BioSystems).

Both programmes allow assignment of values to each DNA fragment for fragment size (a function of speed of migration in the capillary and the number of base-pairs of DNA in the fragment), which following analysis are represented as peaks with their height and area expressed in relative fluorescence units (r.f.u).

### Trial 1

In mixed samples in which every contributor's genotype was present only known exclusive alleles were scored. All other alleles were ignored.

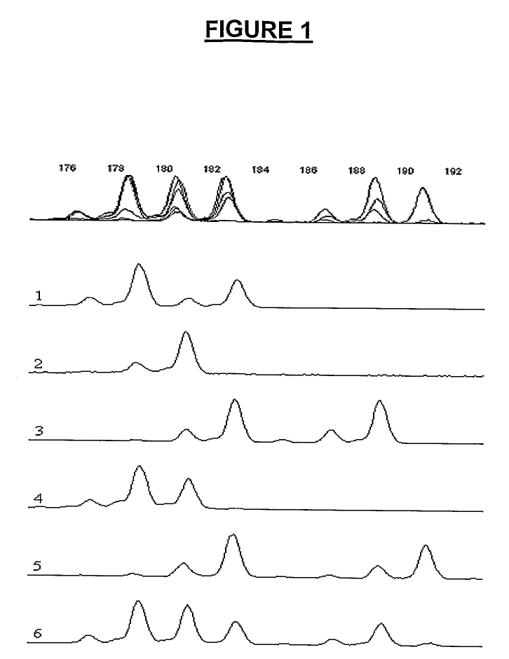

FIG. 1 contains an example of the results of one experiment where the genotypes of five contributing individuals at a typical marker, and the genotype of a mixed sample (row 6) are shown. Individuals in rows 3 and 5 each have an allele that is exclusive within the group.

In the mixture, the presence of these animals can be assumed because both of the exclusive alleles are present, although the exclusive allele of the individual in row 5 (approximately 191 bp) is making a very minor contribution to the mixture.

The individuals in rows 1, 2 and 4 have only common alleles, therefore are unable to be assigned unequivocally, although the genotype of the mixture shows alleles corresponding to each individual.

The inventors did not detect all of the exclusive alleles of any individual in all samples. Apart from one contributor the inventors detected some of the exclusive alleles of each individual in samples 80-100% of the time.

The presence of un-sampled individuals can be inferred by alleles seen in the batch samples but not found in the meat of the contributors sampled. Conversely, any un-sampled individuals that did not have exclusive alleles would have gone unnoticed.

Overall, some exclusive alleles from each animal were seen in all samples, whereas all of the exclusive alleles were only ever seen together in some of the samples.

In a mixture containing three known contributors, one individual had nine exclusive alleles of which some were seen in 80% of samples, but all alleles were never seen together in any sample (s.e.±0.18).

In contrast, another individual had five exclusive alleles of which some were seen in every sample and all were seen together in 80% of samples (s.e.±0).

This demonstrates that mixture profiles can be used to identify individuals when there are a limited number of possible contributors to the mixture.

### Trial 2

The use of a DNA profile of the whole mixture can be used to screen batch samples if the compound meat product being traced cannot be tentatively assigned to only one batch prior to sampling.

To prove the procedure, samples where at least some genotypes were available from known contributors were sub-sampled and each sub-sample visually dissected into individual component particles.

The component particles were genotyped and individual genotypes matched to genotypes of known contributors.

The individual procedure was tested in a blind experiment where the inventors pre-sampled the meat contributing to a batch of meat patties, and were given twelve patties to determine which patties came from the batch.

If more than two alleles were found at any of the loci the sample was scored as contaminated.

The current methodology indicates that uncontaminated muscle fibres from a single individual were obtained at least 50% of the time.

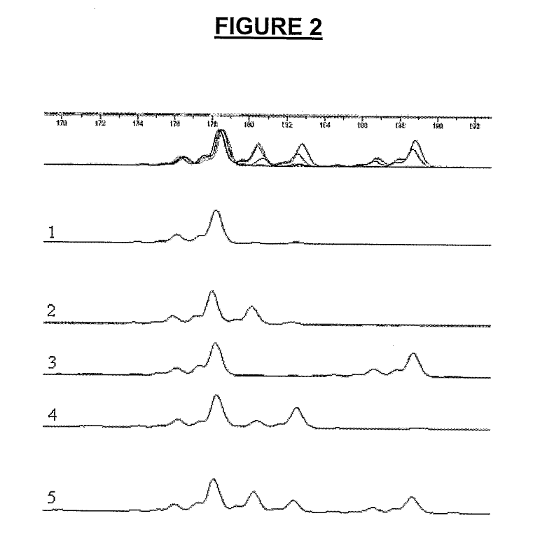

In FIG. 2, examples of genotype profiles at a single locus are shown for five fibres of muscle tissue. Four of the genotypes identify individuals and the fifth is a contaminated sample.

For the individual procedure, the inventors were able to match muscle tissue fibres to six of the seven known contributors to the batch, with each contributor matching up to four fibres from between one and four different patties.

The inventors also found two fibres (with different genotypes) that did not match any of the known contributors, indicating that at least two contributors were not sampled.

From the genotypes matched, the inventors concluded that six of twelve unknown patties had come from the batch that was sampled.

When the inventors checked with the processor, it was confirmed the inventors had correctly identified that six patties had come from the batch and that the six patties nominated were in fact the correct six.

### Trial 3

The use of a DNA profile of the whole mixture can be used to screen batch reference-samples if the compound meat product being traced cannot be tentatively assigned to only one batch prior to sampling.

All patties were sub-sampled and the weight of the sample determined. Analyses were performed on the same weight of material for every sample, and the DNA from every sample was diluted to the same concentration. These are both key points as the procedure is dependant on identifying relative differences between the mixtures.

The reference procedure was tested in two blind experiments, where the inventors were given 18 and 16 reference patties respectively (two reference patties per batch), and were given four and five patties respectively to determine the batch of origin.

For the reference procedure the inventors were able to correctly predict the batch of origin of three of four patties in the first experiment and five of five patties in the second experiment. This included correctly identifying that two of the five patties in the second experiment had come from the same batch.

This experiment also showed that the invention is equally valid for material that is fresh/frozen or cooked/frozen.

### Combined Data

The inventors have undertaken a series of experiments to test the procedures. During each of these tests the production batch of anonymous ground beef patties was correctly identified. In addition, an experiment was conducted whereby patties were labelled as to which batch they had been produced in to test the methodology. In this case some patties were deliberately labelled with the incorrect batch number. The inventors were able to identify which patties had been labelled correctly and which had not.

As a further precaution the data from several experiments were pooled and reanalysed as though it was a single experiment. In this case a total of 16 patties were tested against 40 possible batches. In some cases more than one patty had come from the same batch. The 40 batches had been manufactured at different times over a period of 7 months.

Using the methods described, all 16 test patties were correctly identified to their batch of manufacture. The probability of achieving this result by chance is calculated to be 9.2×10⁻²⁷.

### Discussion

Technical aspects of microsatellites are liable to interfere with analysis (Vignal, Milan, SanCristobal & Eggen; Genetics Selections Evolutions, 34, 275-305, 2002). These can make identifying individual animals within a genotype obtained from a mixture of animals difficult.

An artefact of PCR amplification of microsatellites is stutter. The markers amplify two base pair repeats, so each allele is a minimum of two base pairs from its nearest neighbours. As well as the major allele(s), low amounts of fragments 2, 4 or occasionally 6 base pairs smaller than the actual allele are also amplified.

Microsatellite methodologies are designed for comparing genotypes of individual animals; when an uncontaminated sample from an individual is genotyped there are no more than two major peaks seen at each locus. Major peaks are the real alleles and stutter is a minor consideration because differences in peak height eliminate indecision in allele identification. In a mixture, stutter may mask exclusive alleles that are only making a small contribution to the mixed genotype.

The inventors have previously found it is rare to identify genuine individual allele peaks below 1000 r.f.u. (relative fluorescence units) at the concentration of DNA analysed.

As a guideline, stutter peaks are usually less than 15% of the area of the associated allelic peak. Therefore, any peaks lower than 140 r.f.u. need not be scored as part of the genotype of the mixture.

The profiles of mixtures are generated using a known weight of tissue and a standardised DNA concentration.

The batch reference-sample is subject to the efficiency of mixing, and poor mixing may render the sample useless.

The inventors have shown that it is possible to correctly predict the batch of origin of 3 out of 4 (in one experiment) and 5 out of 5 (in another experiment) by comparing the profile of each sample patty to the average profile of two reference patties from each of a possible 9 and 8 batches respectively.

The inventors have also shown that the predictions are correctly repeated when larger numbers of trace patties and potential batches made over an extended period are combined and analysed as one experiment.

After further refinement of the analyses the inventors have been able to correctly predict the batch of origin of 16 out of 16 patties from a pool of 40 batches.

The inventors have also shown that patties incorrectly labelled for production batch can be identified to the true production batch.

In cases where individual animals from within a mixture were to be identified, only genotypes with one or two alleles at every scored marker were used to identify individuals. If a sample had greater than two alleles even at only one marker, the whole genotype was scored as contaminated.

The inventors have shown that if DNA profiles from at least some (preferably a representative number) of the individuals that contributed to a batch of compound meat product are obtained (be it from database records or from a reference-sample), and that same combination of individuals cannot have also been placed in any other batch, by identifying similar feed contributors to a given sample of compound meat product it is possible to extrapolate that the compound meat product originated from the specified batch.

Present methods of tracing compound biological products for batch recall as a means of quality control currently require an auditable paper-base traceability system to be in place.

Accordingly, a method of DNA traceability would allow independent verification that the correct batch has been recalled or that the products returned in response to a recall are in fact from the appropriate batch. Using current technologies, a recalled batch can only be identified if the product is returned in the original, undamaged packaging. By using DNA traceability techniques, it is possible to unambiguously match compound biological products to the batch of origin, offering the food industry both traceability and quality assurance over those methods presently available.

Aspects of the present invention are described by way of example only and it should be appreciated that modifications and additions may be made thereto without departing from the scope of the appended claims.

1. A method for identifying the batch origin of a compound biological product, comprising the steps of either:
(i) obtaining from a reference-sample at least one genetic profile of at least one individual contributor to a batch of compound biological product; or
(ii) obtaining from a reference-sample a genetic profile of a compound biological product from a batch wherein the genetic profile of individual contributors may or may not be known;
the method further comprising:
a) recording the genetic profiles from i) or ii) to create reference-sample records and linking these to batch information for the purposes of later identification;
b) taking a test-sample of the compound biological product to be identified,
c) optionally, obtaining a component-sample by reducing the test-sample into one or more component particles (i.e. individual contributors);
d) obtaining at least one genetic profile of either the test-sample at step b) or component-samples at step c); and
e) comparing the profile(s) from step d) to the genetic profiles from steps (i) or (ii) of the reference-sample records, for at least one match, so as to identify the batch origin of the compound biological product of the test-sample.

2. The method according to claim 1, wherein the compound biological product refers to any product which includes a component from more than one discrete animal source.

3. The method according to claim 1, wherein the compound biological product is a compound meat product.

4. The method according to claim 1, wherein the genetic profile is information relating to at least one marker.

5. The method according to claim 4, where the genetic profile is information relating to at least 15 markers.

6. The method according to claim 4, wherein at least one marker is a polymorphic microsatellite.

7. The method according to claim 4, wherein at least one marker is an SNP.

8. The method according to claim 1, wherein the reference-sample refers to a sample which can provide a genetic profile indicative of a batch.

9. The method according to claim 8 wherein the reference-sample is indicative of one or more contributors to a batch which are indicative of a batch of origin.

10. The method according to claim 1, wherein batch refers to a defined quantity of compound biological product, identified as being produced from components obtained from exactly the same set of biological sources at a specific time, date and place of manufacture.

11. A method as claimed in claim 1 wherein the genetic profiles are recorded in a database.

12. A method as claimed in claim 11 wherein the genetic profiles are recorded in a computer database.

13. The method according to claim 1, wherein the method includes the further step of applying a mathematical formula using information on genetic inheritance to assign probabilities for the misidentification by DNA analysis of individuals in a sample, due to relatedness of one or more of those individuals in the reference-sample, wherein said mathematical formula to assigning a probability that the profiles of two unrelated individuals matching is
$${MP}_{U} = {\prod }_{j = 1}^{k} [ 2 {( {\sum }_{i = 1}^{{v}_{j}} {p}_{ij}^{2} )}^{2} - {\sum }_{i = 1}^{{v}_{j}} {p}_{ij}^{4} ]$$
Where pᵢⱼ refers to the i^{th} allele of the j^{th} marker;
Vⱼ is the number of alleles of the j^{th} marker, and
k is the number of markers.

14. A method as claimed in claim 1 comprising the further steps of:
iii) inputting the genetic profile information into a computer database, after obtaining a genetic profile from steps (i) or (ii);
f) inputting the genetic profile information into a computer database, after obtaining a genetic profile at step d) and wherein step e), insofar as it relates to a genetic profile of a test sample (as opposed to a component sample) is undertaken by a suitably programmed computer which can access said database.

15. A method as claimed in claim 14 wherein the computer is programmed to assess the posterior probabilities of the sample being derived from each of the reference-sample sources.

16. A method as claimed in claim 15 wherein the computer is programmed to assess the posterior probabilities of the sample being derived from each of the reference-sample sources via a statistical classification assessment.

17. A method as claimed in claim 15 wherein the computer is programmed to assess the posterior probabilities of the sample being derived from each of the reference-sample sources via a supervised machine learning assessment.

18. A method as claimed in claim 1 wherein the number of genetic profiles required to be obtained from individual contributors, where it is envisaged only one component particle will subsequently be available for testing, is determined after assigning a level of probability, of incorrectly failing to identify a match.

19. A method as claimed in claim 18 wherein the assigned level of probability is set at 10⁻⁵.

20. A method as claimed in claim 18 wherein the level of probability is determined by applying the formula:
$$\begin{matrix}P ( {{⋃}_{i = 1}}^{{k}_{r}} {E}_{i} ) = {\sum }_{i = 1}^{{k}_{r}} {E}_{i} - {\sum }_{i < j} \sum {E}_{i} {E}_{j} + {\sum }_{i < j < k} \sum \sum {E}_{i} {E}_{j} {E}_{k} - … \\ = \frac{{k}_{r}}{n} - ( \begin{matrix}{k}_{r} \\ 2\end{matrix} ) \frac{1}{{n}^{2}} + ( \begin{matrix}{k}_{r} \\ 3\end{matrix} ) \frac{1}{{n}^{3}} - … \\ = {\sum }_{u = 1}^{{k}_{r}} - {( - 1 )}^{u} ( \begin{matrix}{k}_{r} \\ i\end{matrix} ) \frac{1}{{n}^{u}}\end{matrix}$$
wherein variable ‘P’ is the probability of the test-sample matching the reference-sample;
and wherein variable ‘n’ is the maximum number of individuals likely to be represented in the batch,
and wherein kᵣ is the number of component particles from the reference samples;
and Ei denotes that there is a match between a test sample and the ith reference sample;
and i, j, k and u are indexing variables associated with the component particles.

21. A method as claimed in claim 1 wherein the number of genetic profiles required from individual contributors, where it is envisaged more than one component particle will be subsequently available for testing, is determined after assigning a level of probability, of correctly identifying a match, after a set number of unique genetic profiles derived from the test sample, are found to correspond to genetic profiles in the reference sample records, for a given batch.

22. A method as claimed in claim 21 wherein the set number is determined according to the estimated number of contributors to a batch and the likelihood of any of those individuals contributing to more than one batch.

23. A method as claimed in claim 21 wherein the probability is set at 0.95.

24. A method as claimed in claim 1 wherein the number of genetic profiles required to be obtained from individual contributors, where it is envisaged that more than one component particle will subsequently be available for testing, is determined after assigning a level of probability, of failing to identify a match.

25. A method as claimed in claim 24 wherein the assigned level of probability is set at 10⁻⁵.

26. A method as claimed in claim 24 where the level of probability is set by the simulation steps of:
1) Generating a list of n contributor identifiers;
2) Generating a list of kᵣ results from the reference product, by sampling (with replacement) from the contributor identifiers;
3) Generating a list of kₜ results from the reference product, by sampling (with replacement) from the contributor identifiers;
4) Counting the number of unique identifiers that appear in both lists generated in steps 2 and 3;
5) Repeating steps 2-4 sufficiently many times to give a stable distribution of the counts in step 4; and
6) Converting the accumulated results from step 4 into proportions.

27. A computer which is programmed to identify the batch origin of a compound biological product from a computer database of reference-sample records linked to batch information via a method comprising:
a) inputting information of at least one genetic profile obtained from a test-sample or component-sample into the computer;
b) comparing the genetic profile(s) from step a) against the appropriate genetic profiles of a reference-sample computer database;
c) calculating likelihoods of a match and converting them to statistical probabilities.

28. A computer storage medium which includes a program to perform a method comprising the steps of either:
(i) obtaining from a reference-sample at least one genetic profile of at least one individual contributor to a batch of compound biological product; or
(ii) obtaining from a reference-sample a genetic profile of a compound biological product from a batch wherein the genetic profile of individual contributors may or may not be known:
the method further comprising:
a) recording the genetic profiles from i) or ii) to create reference-sample records and linking these to batch information for the purposes of later identification;
b) taking a test-sample of the compound biological product to be identified,
c) optionally, obtaining a component-sample by reducing the test-sample into one or more component particles (i.e. individual contributors);
d) obtaining at least one genetic profile of either the test-sample at step b) or component-samples at step c); and
e) comparing the profile(s) from step d) to the genetic profiles from steps (i) or (ii) of the reference-sample records, for at least one match, so as to identify the batch origin of the compound biological product of the test-sample.

29. A method of determining batch origin according to claim 1, wherein prior to steps (i) or (ii), the method further comprises:
a) storing the reference-samples from a batch;
b) linking the reference-samples to batch information; and
c) assigning a level of probability of correctly identifying a match, after a set number of unique genetic profiles derived from the test sample or assigning a level of probability of failing to identify a match.
